(19) United States
(12) Patent Application Publication
(10) Pub. No.: US 2010/0062037 A1
(43) Pub. Date: Mar. 11, 2010

(54) SUBCUTANEOUS IMPLANTS RELEASING AN ACTIVE PRINCIPLE OVER AN EXTENDED PERIOD OF TIME

(76) Inventors: Patrice Mauriac, Paris (FR); Pierre Marion, Neuilly Plaisance (FR)

Correspondence Address: ABELMAN, FRAYNE & SCHWAB, 666 THIRD AVENUE, 10TH FLOOR, NEW YORK, NY, 10017, US

(21) Appl. No.: 12/309,771

(22) PCT Filed: Aug. 1, 2007
(86) PCT No.: PCT/EP2007/057967

§ 371 Date: Aug. 18, 2009

(30) Foreign Application Priority Data
Aug. 2, 2006 (IT) MI2006A001538

(51) Int. Cl. A61K 9/00; B29C 47/00; A61K 38/16; A61K 31/66; A61P 25/00; A61K 31/663

(52) U.S. Cl. 424/423; 264/239; 514/12; 514/103; 514/108

(57) ABSTRACT

Subcutaneous implants obtained by extrusion containing an active ingredient dispersed in a PLGA matrix obtained by grinding an extruded product consisting of a blend of: at least two PLGA having different lactic acid/glycolic acid molar ratios and different weight average molecular weights, or a PLGA and PLA having different weight average molecular weight.

### FIELD OF THE INVENTION

The present invention relates to subcutaneous implants releasing an active principle over an extended period of time, containing an active ingredient dispersed in a polymeric matrix of PLGA, obtained by grinding an extruded product of a blend of at least two PLGA having different lactic acid/glycolic acid molar ratios and different weight average molecular weights, a PLGA and PLA having different weight average molecular weight and the relative process for preparing said implants.

### STATE OF THE ART

Many therapeutic agents are rapidly metabolized and eliminated by the human or mammalian organism, therefore requiring frequent administration of the drug with the aim of maintaining an adequate therapeutic concentration.

The advantage of using implants containing controlled release drugs is well known in the state of the art.

Among the numerous subcutaneous implants known in the art, those described in WO00/33809 represent a net improvement with reference to previous subcutaneous implants containing, as the active principle, a polypeptide dispersed in a matrix of polylactic-glycolic acid in that they are able to release the aforesaid active principle in 6 months. The subcutaneous implants described in said previous patent application differ also in that they present an essentially triphasic and not biphasic release profile, namely a release by pure diffusion, controlled diffusion following swelling and release by polymer degradation

This progression therefore allows for an extension of release times. In fact when these implants are introduced into an aqueous medium, the water diffuses through the polymeric matrix reaching the peptide particles closest to the surface and subsequently the inner zones.

The implant remains substantially unmodified for about 6 weeks and in this period releases approximately 30% of the peptide.

The duration of this stage of pure diffusion is essentially determined by the level of heterogeneity of the peptide dimensions and the rate is essentially determined by the particle content in the PLGA matrix.

As the active principle presents heterogenous particles of dimensions, a sufficient quantity of peptide remains after the first stage of dissolution and can be released in the successive stages mentioned, that is release by diffusion and swelling, and release by disintegration of the polymer.

All the subcutaneous implants including those previously mentioned suffer from a drawback essentially caused by the fact that once the same are administered in the human body, mainly in the first days they release daily a considerably high overall amount of active principle (in some cases decidedly higher than maximum permitted daily dosages).

This is essentially caused by an immediate dissolution of the active principle, this phenomenon, which does not deplete in subsequent days but some times increases in a scalar progression, is known as initial “burst”. In such cases, therefore, it can be verified that the quantity of drug released from such systems, although if compared to the quantity of total active principle contained in the subcutaneous implants administered is low, can in some cases be considered dangerous if with such an initial burst the maximum permitted daily dosage for such a type of drug is approached or exceeded.

Subcutaneous implants having limited initial release of the active principle and consequently a linearly varying release thereof consisting of:

a core (i) comprising an active principle dispersed in a polymeric matrix of polylactic-glycolic acid (PLGA) copolymer,

a coating in film form (ii), essentially consisting of a lactic-glycolic acid copolymer, and the relative processes for preparing said implants are described in WO 2005/000278 A1.

### SUMMARY OF THE INVENTION

The Applicant has now unexpectedly found subcutaneous PLGA based implants formulations which overcome the above drawback, namely lack of linearity of the release profile and important initial release rate or burst release, without the necessity to coat said implants as imposed in the aforementioned WO2005/000278 A1.

The present invention therefore relates to subcutaneous implants obtained by extrusion containing an active ingredient dispersed in a PLGA matrix, wherein said matrix is obtained by grinding an extruded product consisting of a blend of:
at least two PLGA having different lactic acid/glycolic acid molar ratios and different weight average molecular weights, or
a PLGA and PLA having different weight average molecular weight.

Such implants result to release their active ingredient on a fairly linear way or, more generally speaking, in a way that could fit in with a specific set of criteria for a particular formulation purpose (e.g. increasing the overall release duration by limiting the initial burst release).

### DESCRIPTION OF THE FIGURES

FIG. 1 shows, in ordinates, the active ingredient overall release profile expressed in mg versus, in abscissa, the time expressed in days after immersion in the aqueous medium of the subcutaneous implants prepared as described in Example 1.

FIG. 2 shows, in ordinates, the active ingredient overall release expressed in of total, versus, in abscissa, the time expressed in days after immersion in the aqueous medium of the sole subcutaneous implant 1#3 prepared as described in Example 1.

FIG. 3 reports, in ordinates, the active ingredient overall release profile expressed in mg, versus, in abscissa, the time expressed in days after immersion in the aqueous medium of the subcutaneous implants prepared as described in Example 2.

FIG. 4 reports, in ordinates, the active ingredient overall release expressed in mg versus, in abscissa, the time expressed in days after immersion in the aqueous medium of the subcutaneous implants prepared as described in Example 3.

FIG. 5 reports, in ordinates, the active ingredient overall release expressed in mg versus, in abscissa, the time expressed in days after immersion in the aqueous medium of the subcutaneous implants prepared as described in Example 4.

FIG. 6 reports, in ordinates, the active ingredient overall release expressed in mg versus, in abscissa, the time expressed in days after immersion in the aqueous medium of the subcutaneous implants prepared as described in Example 5.

### DETAILED DESCRIPTION OF THE INVENTION

The subcutaneous implants of the present invention preferably contain active principles chosen from the group consisting of: a peptide, an active principle able to increase bone density, an analgesic-narcotic active principle, a steroid hormone, for hormone treatments during menopause and for contraception.

More preferably said peptide is chosen from: avorelin, triptorelin, goserelin and leuprorelin.

The active ingredient able to increase bone density are preferably chosen from: pharmaceutically acceptable bisphosphonic acids and their salts, vitamin D or analogues thereof and sex hormones.

Of these bisphosphonic acids and their pharmaceutically acceptable related salts, we mention for example the compounds of general formula (I):

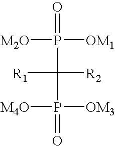

in which M₁, M₂, M₃ and M₄ are monovalent cations and/or H, where said monovalent cations are chosen from alkaline metals, or cations of aliphatic or cycloaliphatic amines, and even more preferably said cations are Na⁺, we would cite for example those in which R₁ and R₂ have the meanings given in the following table 1:

TABLE 1

| Bisphosphonate | R₁ | R₂ |
|---|---|---|
| Etidronate | OH | CH₃ |
| Chlodronate | Cl | Cl |
| Pamidronate | OH | CH₂CH₂NH₂ |
| Alendronate | OH | CH₂CH₂CH₂NH₂ |
| Risedronate | OH | CH₂-3-pyridine |
| Tiludronate | H | CH₂—S-phenyl-4Cl |
| Ibandronate | OH | CH₂CH₂N(CH₃)pentyl |
| Zoledronate | OH | CH₂CH₂-1-imidazole |
| Minodronate | OH | CH₂CH₂-2-imidazopyridinyl |
| Incadronate | OH | N-(cycloheptyl) |
| Olpadronate | OH | CH₂CH₂N(CH₃)₂ |
| Neridronate | OH | CH₂CH₂CH₂CH₂CH₂NH₂ |
| EB1053 | OH | CH₂-1-pyrrolidinyl |

Particularly preferred are disodium etidronate, disodium alendronate and disodium pamidronate.

Preferably the sex hormones are selected from the group of estrogens and progestins and of the latter, androgenic progestins are preferably used.

Estrogens are of steroid type preferably chosen from the class consisting of estradiol, estradiol valerate, estradiol cypionate, estrone, estrone sulphate or estrogens of non-steroidal type for example diethylstilbestrol, p-p′-DDT, bis-phenyl-A.

Among male progestins those preferred are chosen from the class consisting of norethindrone, norethinodrel, norgestrel, desogestrel, norgestimate.

As “drugs with narcotic analgesic activity” preferred are morphine and morphinans, i.e. compounds having a chemical structure and activity similar to that of morphine i.e. μ receptor agonists, but also compounds with morphinic-type activity, in other words also μ receptor agonists but with a different chemical structure such as those belonging to the phenylpiperidine class. (Goodman & Gilman's “The pharmacological basis of therapeutics “Ninth Edition Chapter 23 pages 521-555).

As phenylpiperidine μ receptor agonists we cite as preferred at least one active principle chosen from the class consisting of meperidine, fentanyl and relative pharmaceutically acceptable salts, fentanyl congeners, for example sufentanyl, alfentanyl, lofentanyl, carfentanyl, remifentanyl and their pharmaceutically acceptable salts.

The active principle present in the subcutaneous implants of the invention can present heterogeneous dimensions or can have a more homogeneous particle size distribution.

Preferably, when the subcutaneous implants according to the present invention contain a peptide as the active ingredient, they show a heterogeneous size distribution more preferably ranging from 1 to 63 μm or from 1 to 100 μm.

Specifically, when the subcutaneous implants of the invention contain the peptides having the aforesaid heterogenous particles size dimensions, the resulting extruded blended PLGA contained in the subcutaneous implants according to the present invention has preferably a lactic acid/glycolic acid molar ratio ranging from 50/50 and 90:10 and the weight average molecular weight ranges from 50000 to 150000.

The subcutaneous implants according to the present invention are prepared with a process comprising the following steps
a) Mixing at least two PLGA having different weight average molecular weight and different lactic acid/glycolic acid molar ratio,
b) extruding the powder mix coming from step (a) and then grinding the extruded PLGA mixture, thereby obtaining granules of the blended extruded PLGA
c) dry mixing the active agent in the form of particles with the granules of blended extruded PLGA obtained in step (b) or (c′) wet granulating the particles of said active ingredient and the granules of extruded blended PLGA coming from step (b) in the presence of a suitable solvent, such as water or lower alcohol,
d) drying the granulated product coming from the wet granulation of step (c′) thereby obtaining a residue containing a maximum liquid content of between 0.1 and 3%,
e) extruding the dry mixture obtained in step (c) or the dried granulated product from step (d).

We report herewith for illustrative but not limiting purposes the following examples of the subcutaneous implants according to the present invention.

### Example 1

### Preparation of Subcutaneous Implants Containing Goserelin (Formulations No. 1#1, 1#2 and 1#3)

Subcutaneous implants containing 23.5% w/w Goserelin (having particle size distribution ranging from 1 to 63 μm) and PLGA having compositions, L/G molar ratios and molecular weights as defined in the table below are prepared as described in WO00/33809

| | | Resulting “blended” | |
|---|---|---|---|
| | | PLGA | |
| | | L/G molar | Molecular |
| | PLGA mix composition | ratio | weight |
| 1#1 | 1 single PLGA | 59/41 L/G | 60 kg/mol |
| 1#2 | 2 PLGAs: | | |
| | 25% m/m of a 72/28 L/G - 118 Kg/mol | | |
| | PLGA | | |
| | 75% m/m of a 54/46 L/G - 51 Kg/mol | | |
| | PLGA | | |
| 1#3 | 3 PLGAs: | | |
| | 37.5% m/m of a 72/28 L/G - 118 Kg/mol | | |
| | PLGA | | |
| | 37.5% m/m of a 54/46 L/G - 51 Kg/mol | | |
| | PLGA | | |
| | 25% m/m of 51/49 L/G - 17 Kg/mol | | |

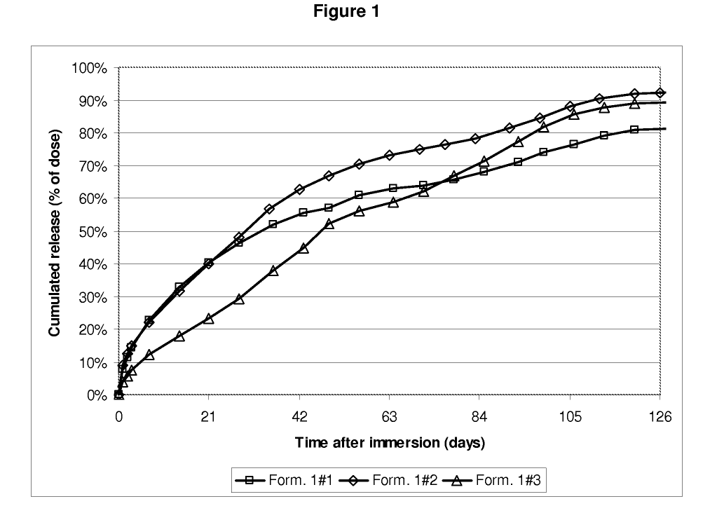

FIG. 1 shows, in ordinates, the active ingredient overall release profile expressed in mg versus, in abscissa, the time expressed in days after immersion in the aqueous medium of the subcutaneous implants in the aqueous medium prepared as described in Example 1.

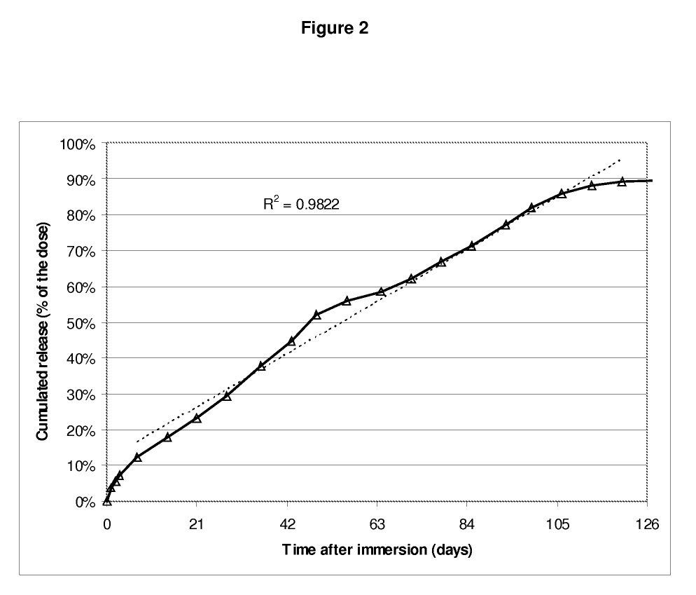

FIG. 2 shows, in ordinates, the active ingredient overall release profile expressed in % of total, versus, in abscissa, the time expressed in days after immersion in the aqueous medium of the sole subcutaneous implants 1#3 prepared as described in Example 1.

It is observed from FIG. 1 that slower release profiles are obtained in the first 3-4 weeks with the subcutaneous implants 1#2 and 1#3 containing the blended extruded PLGA having a resulting weight average molecular weight of 60000 Da and an L/G ratio of 59/41 if compared to that obtained with subcutaneous implants MedRH108 containing a single PLGA having the same and aforementioned average molecular weight and L/G molar ratio values of the resulting blended PLGA utilised in 1#2 and 1#3. In this case, the formulation (1#3) actually leads to a more fairly linear release profile (R²=0.9820 for % active ingredient released=f(t) from week 1 to 17 calculated by linear regression—see FIG. 2).

### Example 2

### Preparation of Subcutaneous Implants Containing Leuprorelin (Formulations No. 2#1 and 2#2)

Subcutaneous implants containing 23.5% w/w Leuprorelin (having particle size distribution ranging from 1 to 100 μm) and PLGA having compositions, L/G molar ratios and molecular weights as defined in the table below are prepared as described in WO00/33809

| | | Resulting “blended” | |
|---|---|---|---|
| | | PLGA | |
| | | L/G molar | Molecular |
| | PLGA mix composition | ratio | weight |
| 2#1 | 1 single PLGA | 75/25 L/G | 110 kg/mol |
| 2#2 | 2 PLGAs: | 70/30 L/G | ~90 kg/mol |
| | 75% m/m of a 75/25 L/G - 110 Kg/mol | | |
| | PLGA | | |
| | 25% m/m of a 51/49 L/G - 18 Kg/mol | | |
| | PLGA | | |

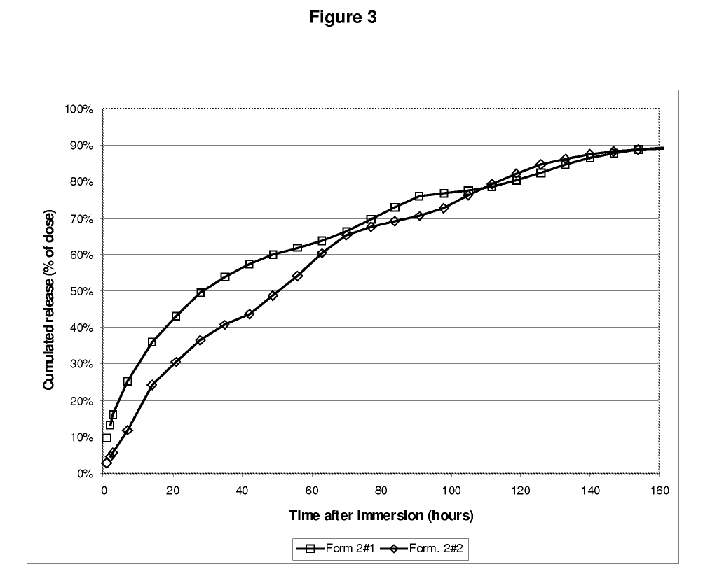

FIG. 3 shows, in ordinates, the active ingredient overall release profile expressed in mg, versus, in abscissa, the time expressed in days after immersion in the aqueous medium of the subcutaneous implants prepared as described in Example 2, and in the smaller diagram the active ingredient release profile expressed in mg in the first seven days of release.

It is observed that the addition of a higher Glycolide ratio and lower molecular weight PLGA actually leads to lower initial release rate and to a more linear release profile when compared to the one obtained with a single 75/25—high molecular weight PLGA.

### Example 3

### Preparation of Subcutaneous Implants Containing Leuprorelin (Formulations No. 3#1 and 3#2)

Subcutaneous implants containing 27% w/w Leuprorelin (having particle size distribution ranging from 1 to 100 μm) and PLGA having compositions, L/G molar ratios and molecular weights as defined in the table below are prepared as described in WO00/33809

| | | Resulting “blended” | |
|---|---|---|---|
| | | PLGA | |
| | | L/G molar | Molecular |
| | PLGA mix composition | ratio | weight |
| 3#1 | 2 PLGAs: | 70/30 L/G | 87 kg/mol |
| | 75% m/m of a 75/25 L/G - 110 Kg/mol | | |
| | PLGA | | |
| | 25% m/m of a 51/49 L/G - 18 Kg/mol | | |
| | PLGA | | |
| 3#2 | 2 PLGAs: | 80/20 L/G | ~90 kg/mol |
| | 75% m/m of a 75/25 L/G - 110 Kg/mol | | |
| | PLGA | | |
| | 25% m/m of a 100/0 L/G - 15 Kg/mol | | |
| | PLGA | | |

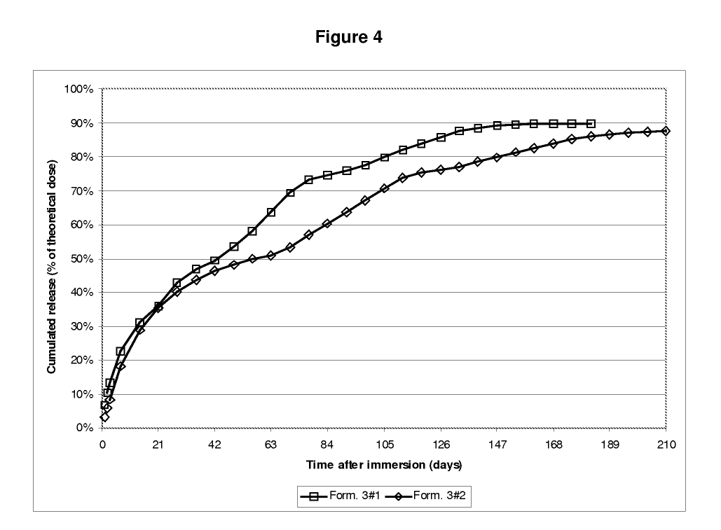

FIG. 4 shows, in ordinates, the active ingredient overall release profile expressed in mg versus, in abscissa, the time expressed in days after immersion in the aqueous medium of the subcutaneous implants prepared as described in Example 3. It is observed that the addition of a lower molecular weight PLGA actually leads to lower initial release rate and to longer release duration when compared to the one obtained when adding PLGA with low weight average molecular weight and 51/49 L/G PLGA to the PLGA having a L/G of 75/25 and high molecular weight.

### Example 4

### Preparation of Subcutaneous Implants Containing Fentanyl Citrate

Subcutaneous implants containing 40.0% m/m Fentanyl citrate (having particle size distribution ranging from 1 to 63 μm) and PLGA having compositions, L/G molar ratios and molecular weights as defined in the table below are prepared as described in WO00/33809

| | | Resulting “blended” | |
|---|---|---|---|
| | | PLGA | |
| Formulation | | L/G molar | Molecular |
| Nr | PLGA mix composition | ratio | weight |
| 4#1 | 1 single PLGA | 54/46 | 51 kg/mol |
| 4#2 | 2 PLGAs: | 53/47 | ≈42 kg/mol |
| | 75% m/m of a 54/46 L/G - | | |
| | 51 Kg/mol PLGA | | |
| | 25% m/m of a 50/50 L/G - | | |
| | 17 Kg/mol PLGA | | |

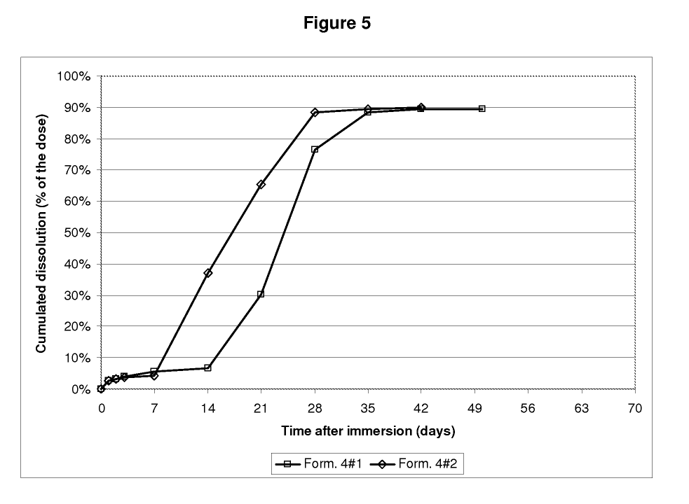

FIG. 5 shows the active ingredient release profiles (% of the dose released versus time after immersion) of the various implants of Example 4.

This example demonstrates that, if compared to the release profile single PLGA (Form. 4#1), the formulation (Form. 4#2) allows to achieve a more linear release profile also between the seventh and 25^{th} day.

### Example 5

### Preparation of Subcutaneous Implants Containing Medroxy Progesterone Acetate

Subcutaneous implants containing 55% m/m Medroxy Progesterone Acetate (having particle size distribution ranging from 1 to 63 μm) and PLGA having compositions, L/G molar ratios and molecular weights as defined in the table below are prepared as described in WO00/33809

| | | Resulting “blended” | |
|---|---|---|---|
| | | PLGA | |
| Formulation | | L/G molar | Molecular |
| Nr | PLGA mix composition | ratio | weight |
| 5#1 | 1 single PLGA | 75/25 | 120 kg/mol |
| 5#2 | 2 PLGAs: | 67.5/32.5 | ≈85 kg/mol |
| | 50% m/m of a 75/25 L/G - | | |
| | 120 Kg/mol PLGA | | |
| | 50% m/m of a 60/40 L/G - | | |
| | 53 Kg/mol PLGA | | |

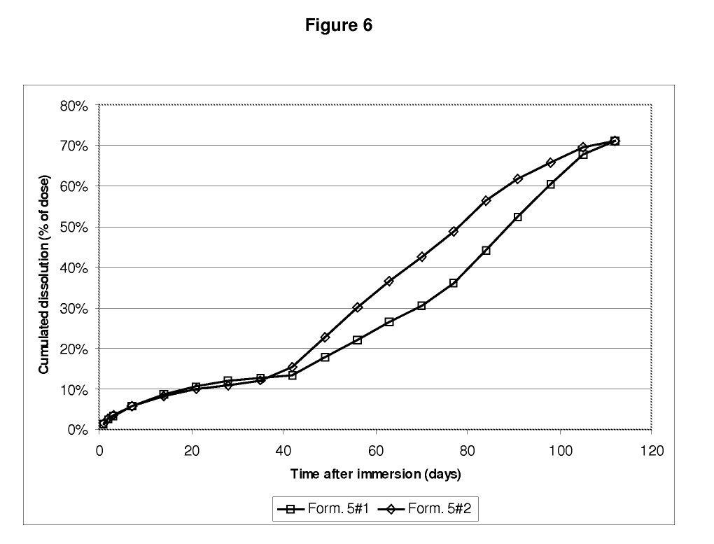

FIG. 6 shows the active ingredient release profiles (% of the dose released versus time after immersion) of the various implants from example 5.

As it results from a comparison of the two release profiles it results that the formulation 5# 2 also after the 40th day of the invention a more linear release profile can be achieved with the subcutaneous implants containing a mixture of PLGA than with the subcutaneous implants containing a single PLGA, having a similar L/G molar ratio and an approximately similar average molecular weight.

1.-13. (canceled)

14. Subcutaneous implants obtained by extrusion containing an active ingredient dispersed in a PLGA matrix, wherein said matrix is obtained by grinding an extruded product consisting of a blend of:
at least two PLGA having different lactic acid/glycolic acid molar ratios and different weight average molecular weights, or
a PLGA and PLA having different weight average molecular weight,
wherein said active ingredient is selected from the group consisting of a peptide, an active ingredient able to increase bone density selected from the group consisting of pharmaceutically acceptable bisphosphonic acids and their salts, vitamin D or analogues thereof and sex hormones, an analgesic-narcotic active principle, a steroid hormone for hormone treatments during menopause and for contraception.

15. Subcutaneous implants according to claim 14 wherein the peptide is selected from the group consisting of avorelin, triptorelin, goserelin and leuprorelin.

16. Subcutaneous implants according to claim 14 wherein the biphosphonic acid salts are selected from the group consisting of disodium etidronate, disodium alendronate and disodium pamidronate.

17. Subcutaneous implants according to claim 14, wherein said sex hormones are selected from the group consisting of estrogens and androgenic progestins.

18. Subcutaneous implants according to claim 17, wherein said estrogens are selected from the group consisting of estradiol, estradiol valerate, estradiol cypionate, estrone, estrone sulphate or estrogens of non-steroidal type.

19. Subcutaneous implants according to claim 17 wherein said androgenic progestins are selected from the group consisting of norethindrone, norethinodrel, norgestrel, desogestrel and norgestimate.

20. Subcutaneous implants according to claim 14, wherein the active ingredient with narcotic analgesic activity is selected from the group consisting of morphine and morphinans, and μ receptor agonists.

21. Subcutaneous implants according to claim 20, wherein said μ receptor agonists are phenylpiperidines selected from the group consisting of meperidine, fentanyl and relative pharmaceutically acceptable salts, fentanyl congeners selected from sufentanyl, alfentanyl, lofentanyl, carfentanyl, remifentanyl and their pharmaceutically acceptable salts.

22. Subcutaneous implants according to claim 14 wherein the active ingredient therein contained has homogeneous or heterogeneous particles size distribution.

23. Subcutaneous implants according to claim 22, wherein when the active ingredient is a peptide, it exhibits a heterogeneous particle size distribution between 1 and 100 μm.

24. Subcutaneous implants according to claim 22, wherein when the active ingredient is a peptide, it exhibits a heterogeneous particle size distribution between 1 and 63 μm.

25. Subcutaneous implants according to claim 23, wherein the resulting extruded blended PLGA has a lactic acid/glycolic acid molar ratio ranging from 50/50 and 90:10 and the weight average molecular weight ranges from 50000 to 150000.

26. A process for preparing the subcutaneous implants according to claim 14 comprising the following steps
a) mixing at least two PLGA having different weight average molecular weight and different lactic acid/glycolic acid molar ratios,
b) extruding the powder mix coming from step (a) and then grinding the extruded PLGA mixture, thereby obtaining the blended extruded PLGA granules,
c) dry mixing the active agent in the form of particles with the granules of said blended extruded PLGA coming from step (b), or (c′) wet granulating the active ingredient particles and the granules of the blended extruded PLGA coming from step (b), using a suitable solvent,
d) drying the granulated product coming from wet granulation of step (c) thereby obtaining a residue containing a maximum liquid content of between 0.1 and 3%,
e) extruding the dry mixture coming from step (c) or the dried granulated product from step (d).
